(19) United States
(12) Patent Application Publication
(10) Pub. No.: US 2004/0236413 A1
(43) Pub. Date: Nov. 25, 2004

(54) Stents with vitronectin receptor antagonists against restenosis

(76) Inventors: Klaus Urbahns, Kyoto (JP); Christoph Gerdes, Leverkusen (DE); Susanne Zuleger, Koln (DE); Sabine Gelfert-Peukert, Wuppertal (DE); Jeffrey Low, Dusseldorf (DE); Jochen Kalbe, Leichlingen (DE)

Correspondence Address: Jeffrey M Greenman, Bayer Corporation, 400 Morgan Lane, West Haven, CT, 06516, US

(21) Appl. No.: 10/484,414

(22) PCT Filed: Jul. 8, 2002
(86) PCT No.: PCT/EP02/07568

(30) Foreign Application Priority Data
Jul. 19, 2001 (DE) 101 35 315.4

(51) Int. Cl. A61F 2/06

(52) U.S. Cl. 623/1.42; 623/1.46

(57) ABSTRACT

The application relates to vitronectin receptor antagonist-containing stents and to their production and use.

Coronary diseases caused by arteriosclerosis are treated inter alia by the currently usual method of percutaneous transluminal coronary angioplasty (PTCA). For this purpose, a balloon catheter is introduced into the narrowed or blocked artery, the latter is widened through expansion of the balloon, and the blood flow is thus restored. In such cases, the acute reocclusion, occurring immediately after the PTCA (acute restenosis), or the later reocclusion (subacute restenosis), of the blood vessel is a problem occurring in about 30% of cases.

The risk of acute restenosis can be reduced by administering platelet aggregation inhibitors. It is also possible for a mechanical support of the coronary wall by a normally cylindrical and expandable mesh (stent), which is introduced into the diseased vessel and unfolds at the site of the stenosis in order to open the narrowed vessel and keep it open by supporting the blood vessel wall, to reduce the risk of restenosis somewhat. However, a satisfactory therapy of subacute restenosis is not currently available.

A more recent possibility for restenosis treatment is the local administration of the active ingredient through use of an active ingredient-releasing stent. This combination of active ingredient and stent achieves medical treatment and mechanical stabilization in one application.

It is possible for this purpose to coat stents with active ingredient-containing coating materials. The active ingredient is released through diffusion from the coating or else through degradation of the coating when biodegradable coating systems are used.

Another possibility which has already been described is the preparation of small cavities or micropores in the stent surface, into which the active ingredient or else active ingredient-containing polymeric coating systems are embedded (EP 0 950 386). It is subsequently possible to apply an active ingredient-free coating. Release takes place through diffusion or degradation or through a combination of the two processes.

It is additionally possible to produce active ingredient-containing stents by melt embedding of the active ingredient in a polymeric carrier, e.g. with the aid of injection moulding processes. Release of the active ingredient from these stents usually takes place by diffusion.

It has now been found, surprisingly, that vitronectin receptor antagonists are particularly suitable as active ingredients for this type of treatment.

The present invention therefore describes the use of one or more vitronectin receptor antagonists for producing a medicinal substance-containing release system, in particular a medicinal substance-containing stent, and a vitronectin receptor antagonist-containing release system, in particular a vitronectin receptor antagonist-containing stent, which makes targeted liberation of the vitronectin receptor antagonist at the site of action possible (drug targeting).

The present invention likewise describes a method for a treatment of restenosis, where one or more vitronectin receptor antagonists are used in combination with a stent. In this application it is possible for the vitronectin receptor antagonist to be employed either systemically or preferably in the form of a vitronectin receptor antagonist-containing stent.

Whereas sufficiently successful therapy cannot be achieved in all cases with the currently available active ingredients and stents, the novel combination of vitronectin receptor antagonists with a stent makes effective treatment of restenosis possible. Local administration of vitronectin receptor antagonists in combination with a stent allows the dose of the medicinal substance necessary to prevent restenosis to be reduced. It is thus possible to minimize unwanted systemic effects. At the same time, the local concentration can be raised and thus the efficacy increased.

Systemic and/or local administration of active ingredients suitable for the prophylaxis or therapy of restenosis, such as, by way of example and preferably, abciximab, eptifibatide, tirofiban, acetylsalicylic acid, ticlopidine or clopidogrel, is of course possible—in addition to the administration according to the invention. An additional systemic treatment with vitronectin receptor antagonists, in particular oral administrations, is preferred.

Vitronectin receptor antagonists mean compounds which block the α₁β₃ and/or the αᵥβ₅ receptor. The compounds preferably used according to the invention as vitronectin receptor antagonists are those described in WO-00/035,864, WO-00/035,917 and WO-00/041,469. The compounds mentioned in general therein, and especially the compounds specifically mentioned therein, expressly form part of the description of the present invention.

Preferred vitronectin receptor antagonists are compounds of the general formula (1)

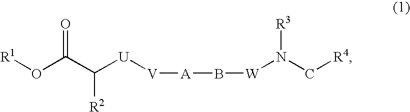

where

R¹ is hydrogen, a substituted or unsubstituted alkyl or cycloalkyl radical, a substituted or unsubstituted aryl radical or a saturated or unsaturated, optionally substituted heterocyclic radical;

R² is hydrogen, a substituted or unsubstituted alkyl or cycloalkyl radical, a substituted or unsubstituted aryl radical, a saturated or unsaturated, optionally substituted heterocyclic radical, an optionally substituted alkenyl radical, an optionally substituted alkynyl radical, —NR^{2′}SO₂R^{2″}, —NR^{2′}COOR^{2″}, —NR^{2′}COR^{2′}, —NR^{2′}CONR_{2′}₂ or —NR^{2′}CSNR^{2′}₂;

R^{2′} is hydrogen, a substituted or unsubstituted alkyl or cycloalkyl radical, a substituted or unsubstituted aryl radical or a saturated or unsaturated, optionally substituted heterocyclic radical;

R^{2″} is a substituted or unsubstituted alkyl or cycloalkyl radical, a substituted or unsubstituted aryl radical or a saturated or unsaturated, optionally substituted heterocyclic radical;

U is a direct linkage or a substituted or unsubstituted alkylene group;

V is a substituted or unsubstituted alkylene group, —NR^{2′}CO— or —NR^{2′}SO₂—;

A and B are each independently of one another a 1,3- or 1,4-bridging, optionally additionally substituted phenylene group;

W is a direct linkage or a substituted or unsubstituted alkylene group;

C is a direct linkage or

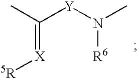

R³ is hydrogen, a substituted or unsubstituted alkyl or cycloalkyl radical, a substituted or unsubstituted aryl radical, a saturated or unsaturated, optionally substituted heterocyclic radical, an alkylamine residue, an alkylamide residue or is connected to one of R⁴, Y, R⁵ or R⁶, if present, to form an optionally substituted heterocyclic ring system which includes the nitrogen atom to which R³ is bonded, and may be saturated or unsaturated and/or comprise further heteroatoms;

R⁴ is hydrogen, a substituted or unsubstituted alkyl or cycloalkyl radical, a substituted or unsubstituted aryl radical, a saturated or unsaturated, optionally substituted heterocyclic radical, an alkylamine residue, an alkylamide residue or is connected to one of R³, Y, R⁵ or R⁶, if present, to form an optionally substituted heterocyclic ring system which includes the nitrogen atom to which R⁴ is bonded, and may be saturated or unsaturated and/or comprise further heteroatoms;

X is CHNO₂, CHCN, O, N or S;

Y is a direct linkage or an optionally substituted alkylene or alkyne group;

R⁵ is absent, hydrogen, a substituted or unsubstituted alkyl or cycloalkyl radical, —NO₂, —CN, —COR^{5′}, —COOR^{5′}, or is connected to one of R³, Y, R⁴ or R⁶, if present, to form an optionally substituted carbocyclic or heterocyclic ring system which includes X, and may be saturated or unsaturated and/or comprise further heteroatoms;

R^{5′} is hydrogen, a substituted or unsubstituted alkyl or cycloalkyl radical, a substituted or unsubstituted aryl radical or a saturated or unsaturated, optionally substituted heterocyclic radical which may be saturated or unsaturated and/or comprise further heteroatoms;

R⁶ is hydrogen, a substituted or unsubstituted alkyl or cycloalkyl radical, a substituted or unsubstituted aryl radical, a saturated or unsaturated, optionally substituted heterocyclic radical, an alkylamine residue, an alkylamide residue or is connected to one of R³, R⁴, Y or R⁵, if present, to form an optionally substituted heterocyclic ring system which includes the nitrogen atom to which R⁶ is bonded, and may be saturated or unsaturated and/or comprise further heteroatoms.

The vitronectin receptor antagonist-containing release systems of the invention are produced by using conventional stents, where the basic body of the stent consists either of metals or undegradable plastics such as, by way of example and preferably, polyethylene, polypropylene, polycarbonate, polyurethane and/or polytetrafluoroethylene (PTFE). In addition, stents with various designs of the metal mesh which make various surfaces and folding principles possible and as described, for example, in WO 01/037761, WO 01/037892 are used as basic bodies of the stents.

These stents are coated and/or filled with the vitronectin receptor antagonists. An alternative possibility in the case of nonmetallic stents is for vitronectin receptor antagonists to be incorporated directly into the material used to produce the stents.

For the coating or filling, carrier materials are mixed with the vitronectin receptor antagonists. The carrier materials preferably used in these cases are polymeric carriers, in particular biocompatible, non-biodegradable polymers or polymer mixtures such as, by way of example, and preferably, polyacrylates and copolymers thereof such as, by way of example and preferably, poly(hydroxyethyl)methyl-methacrylates; polyvinylpyrrolidones; cellulose esters and ethers; fluorinated polymers such as, by way of example and preferably, PTFE; polyvinyl acetates and copolymers thereof; crosslinked and noncrosslinked polyurethanes, polyethers or polyesters; polycarbonates; polydimethylsiloxanes. Also used alternatively as polymeric carriers are biocompatible, biodegradable polymers or polymer mixtures such as, by way of example and preferably, polymers or copolymers of lactide and glycolide, or of caprolactone and glycolide; other polyesters; polyorthoesters; polyanhydrides; polyamino acids; polysaccharides; polyiminocarbonates; polyphosphazenes and poly(ether-ester) copolymers.

Further suitable polymeric carriers are also mixtures of biodegradable and/or non-biodegradable polymers. The rate of release of active ingredient is adjusted optimally by these mixtures.

Coated or filled stents are produced by dissolving the mixtures of vitronectin receptor antagonists and carrier—preferably in suitable solvents. These solutions are then applied to the stent by various techniques such as, for example, spraying, dipping or brush-coating. Subsequent or simultaneous removal of the solvent thus results in the stent provided with active ingredient-containing coating. An alternative possibility is also for mixtures of vitronectin receptor antagonists and carrier to be melted and applied by the same application methods.

The stents are preferably pretreated in order to increase the external and/or internal surface area of the stent. This increases the loading potential, and larger amounts of coating (vitronectin receptor antagonist/polymer) can be applied. Various etching techniques or else treatments with ionizing radiation are used for example for pretreatment of the stents. It is likewise possible to create micropores or cavities in the stents with the aid of various techniques.

The active ingredient contents of the stents coated or filled with vitronectin receptor antagonists are ordinarily from 0.001% by weight to 50% by weight, preferably from 0.01% by weight to 30% by weight, particularly preferably 0.1% by weight to 15% by weight.

In the case of nonmetallic stents, the vitronectin receptor antagonists can also be incorporated directly into the basic body of the stent for example as melt embedding. This involves active ingredient-containing polymeric carrier compositions being processed to the final active ingredient-containing form by conventional processes, for example by injection moulding processes. In this case, the active ingredient is ordinarily released by diffusion.

The active ingredient contents of stents with embedded vitronectin receptor antagonists is ordinarily from 0.001% by weight to 70% by weight, preferably from 0.01% by weight to 50% by weight, particularly preferably 0.1% by weight to 30% by weight.

The vitronectin receptor antagonist-containing stents are additionally coated where appropriate with a membrane. This membrane serves, by way of example and preferably, to control the release of medicinal substance and/or to protect the active ingredient-containing stent from external influences.

1. A stent comprising one or more vitronectin receptor antagonists.

2. A stent for treatment of restenosis following PCTA, comprising one or more vitronectin receptor antagonists.

3. The stent according to claim 1, further comprising an additional membrane coating.

4. The stent according to claim 1, 2, or 3, comprising at least one further active ingredient.

5. A method for the treatment or prevention of reocclusion in restenotic arteries comprising using a vitronectin receptor antagonist-containing stent.

6. A process for production of stents comprising using one or more vitronectin receptor antagonists in the production process.

7. A process for the production of stents, characterized in that stents are coated or filled with one or more vitronectin receptor antagonists.

8. A process for the production of stents, characterized in that a polymeric carrier composition comprising one or more vitronectin receptor antagonists is shaped into a stent.

9. A method for the treatment of patients with restenotic arteries comprising using simultaneously one or more vitronectin receptor antagonists and a stent.

10. The method according to claim 9, characterized in that one or more vitronectin receptor antagonists are present in or on the stent and are released locally.

11. A process for production of stents for the treatment or prevention of restenosis, comprising using one or more vitronectin receptor antagonists in the production process.

12. A method for the prophylaxis or therapy of restenosis through use of a stent according to claim 1, 2, 3, or 4, in combination with local and/or systemic administration of at least one other active ingredient suitable for the prophylaxis or therapy of restenosis.

13. A method for the prophylaxis or therapy of restenosis comprising using a stent according to claim 1, 2, 3, or 4 in combination with systemic administration of a vitronectin receptor antagonist.
